(19) United States
(12) Patent Application Publication
(10) Pub. No.: US 2015/0299768 A1
(43) Pub. Date: Oct. 22, 2015

(54) AN IMPROVED DEVICE AND METHOD FOR SAMPLE SEPARATION

(71) Applicant: JN MEDSYS PTE LTD, Singapore (SG)

(72) Inventors: Kian Kok Johnson NG, Singapore (SG); Koon Kiat TEU, Singapore (SG)

(73) Assignee: JN MEDSYS PTE LTD, Singapore (SG)

(21) Appl. No.: 14/434,069

(22) PCT Filed: Oct. 8, 2013
(86) PCT No.: PCT/SG2013/000433

§ 371 Date: Apr. 7, 2015

(30) Foreign Application Priority Data
Oct. 9, 2012 (SG) 201207579-2

(51) Int. Cl. C12Q 1/68; B01L 3/00; G01N 1/14

(52) U.S. Cl. CPC C12Q 1/6806; G01N 1/14; C12Q 1/686; B01L 3/502; G01N 2001/149; B01L 2300/0819; B01L 2300/0838; B01L 2300/0893; B01L 2400/0406

(57) ABSTRACT

A device comprising: a capillary array of bundled micro-capillary wells; said bundle arranged into a close packed arrangement; a first end of said array forming a sample receiving surface; wherein said device is arranged to draw said sample into said capillary array through capillary action, so as to divide the sample into a plurality of sub-reactions in said wells.

### FIELD OF THE INVENTION

The invention relates to a method and device for the separation of samples into a plurality of sub-samples. In particular, the invention relates to a method arranged to divide said sample into sub-reactions, such as for the purposes of conducting a PCR procedure.

### BACKGROUND

Digital PCR (dPCR) is a powerful and emerging technology in the hugely lucrative PCR market. PCR, particularly real-time PCR, is an indispensable process in many areas of biomedical research and diagnostics and is the most sensitive method for detecting nucleic acids targets such as RNA and DNA. Real-time PCR's popularity lies in its ability to quantify the amount of DNA detected which is important in areas such as cancer diagnostics. Digital PCR combines the quantitative ability of real-time PCR with the simplicity of end-point PCR. It is also extremely sensitive, due to its ability to detect down to even a single DNA molecule, making it particularly useful in certain applications, e.g. detecting genetic aberrations of foetal DNA in maternal plasma which can lead to anon-invasive prenatal test for Downs' Syndrome.

To perform dPCR, a typical PCR solution is partitioned into a large number of very small sub-reactions, such that each sub-reaction has at most a single copy of DNA. After performing PCR, some of the zones will be positive for PCR products, while the others will not, providing a 1 or 0 result, hence the term “digital”. Using Poisson distribution, the amount of starting DNA present can then be quantified. As compared to real-time PCR, which quantifies the DNA by monitoring the PCR temporally (hence the term “real-time”) along with a control for calibration, dPCR performs the quantification by segmenting the reaction into miniscule volumes and monitoring them spatially, without the need for a calibration control.

The sensitivity and precision of dPCR is solely dependent on its ability to partition a PCR sample into thousands of smaller reactions. A larger number of sub-reactions, and a smaller volume per sub-reaction, will enhance the sensitivity and precision.

### SUMMARY OF INVENTION

In a first aspect the invention provides a device comprising: a capillary array of bundled micro-capillary wells; said bundle arranged into a close packed arrangement; a first end of said array forming a sample receiving surface; wherein said device is arranged to draw said sample into said capillary array through capillary action, so as to divide the sample into a plurality of sub-reactions in said wells.

In a second aspect the invention provides a method of placing a sample within a capillary array, said method comprising the steps of: placing the sample on a sample receiving surface of said capillary array; sliding a distribution tool across the sample receiving surface, and so; distributing the sample across the surface, and consequently; drawing said sample into wells of said micro-capillary array through capillary action.

For example, a glass micro-capillary array will be described. It will be appreciated that, for the purposes of the present invention, other materials are equally applicable, including a polymer such as polycarbonate.

It follows that, when a solution sample is added onto one surface of the micro-capillary array, it comes into contact with the micro-capillary wells beneath it. Instantly, these wells draw the solution in as a result of capillary action, which is the tendency of a liquid to be drawn into narrow tubes as a result of adhesion and surface tension. As the micro-capillary wells are very narrow, the capillary action is strong enough to draw the solution in to fill up the entire well. A large number of these wells are filled simultaneously due to the capillary action. The end result is that the solution is partitioned into an array of smaller sub-reactions. When assembled into a casing, such as a tube having a selectively sealable cap, this forms the device of the present invention.

The device may also include a distribution tool, or simply a slide, arranged to slide across said surface to form a film of said sample on said surface. The distribution tool may engage the sample through surface tension that is bringing the tool into contact with the sample with the surface tension attractive forces attaching to the distribution tool. Then, on sliding the tool across the surface, the sample is “pulled” across the surface to bring the sample into contact with a greater proportion of wells, and so increase the number of available sub-reactions. Alternatively, the distribution tool may push the sample across the surface and so distribute the sample across the surface to engage a greater proportion of wells.

### BRIEF DESCRIPTION OF DRAWINGS

It will be convenient to further describe the present invention with respect to the accompanying drawings that illustrate possible arrangements of the invention. Other arrangements of the invention are possible and consequently, the particularity of the accompanying drawings is not to be understood as superseding the generality of the preceding description of the invention.

FIGS. 1A and 1B are schematic views of a micro-capillary array for a device according to one embodiment of the present invention;

FIG. 1C is an isometric view of a micro-capillary array for a device according to a further embodiment of the present invention;

FIG. 2A are sequential views of a distribution tool according to one embodiment of the present invention;

FIG. 2B is a plan view of the distribution tool of FIG. 2A;

FIG. 3A is a sequential elevation view of a distribution tool according to a further embodiment of the present invention;

FIG. 3B is a plan view of the distribution tool of FIG. 3A;

FIGS. 4A and 4B are various views of devices according to further embodiments of the present invention;

FIG. 5A to 5C are various views of devices according to further embodiments of the present invention.

### DETAILED DESCRIPTION

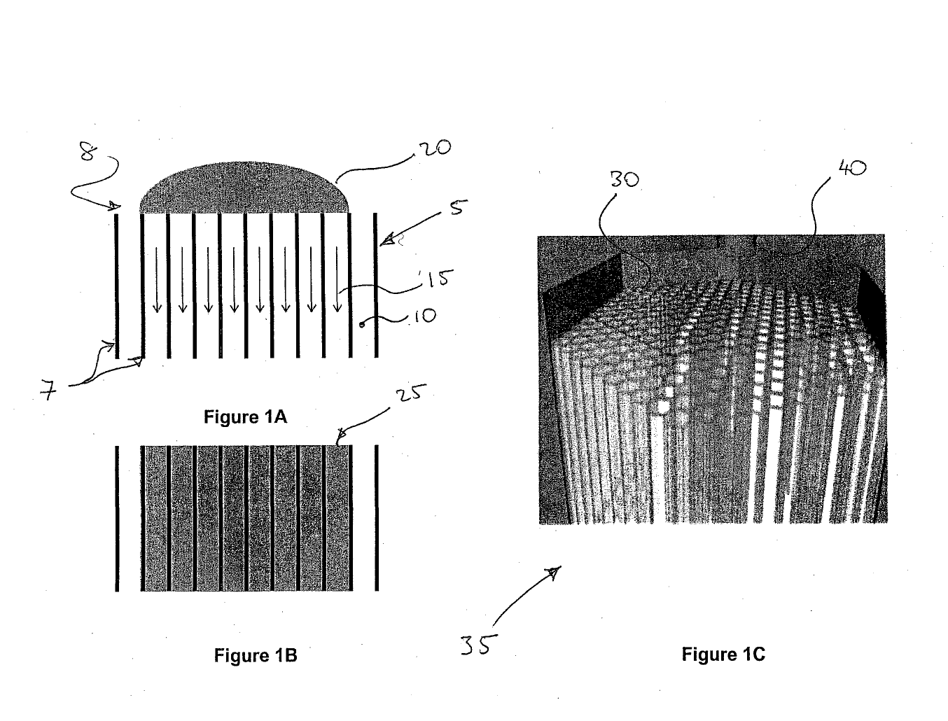

FIGS. 1A and 1B show the general principle of the present invention. Here a micro-capillary array, such as a glass micro-capillary array (GCA) 5 comprises a bundle of micro-capillary wells 7 which when arranged provide a surface 8 upon which a sample 20 can be placed. The micro-capillary wells 7 are sized to draw down 15 the sample 20 into the wells 10 of the wells 7. The result as shown in FIG. 1B is to entrap the sample into sub-reactions 25 within the GCA 5 for subsequent processing. An actual embodiment can be seen in FIG. 1C whereby the device 35 comprises the GCA 30 housed within a case 40. By way of example, each well may be of 100 microns in diameter which may be further drawn down to 10 microns. The purpose of using glass is the ability to draw down the wells to a size to achieve the required volume within each well which is most easily done by glass.

The bundle is arranged in a high density arrangement, such as in a close packed hexagonal arrangement. The bundling of each well into the GCA arrangement may provide an open area ratio that is the ratio of well bore to total area of 80%. The GCA within the device may still be effective within open area ratio down to 30%. The GCA may have a depth of 1 mm or longer subject to the required volume of each well. Thus, for a 1 mm depth each 10 micron well will have a volume of 0.1 nano litres.

For instance, a capillary array may contain at least 200 wells, and therefore up to 200 sub-reactions. However, a more useful embodiment of the present invention may include as many as 5000 wells, and consequently aim for up to 5000 sub-reactions. It may also be possible to create even larger capillary arrays, such as 10,000 or even 100,000 wells.

Each well may have a maximum volume of 50 nl. As calculated above, a useful volume may be as small as 0.1 nl, with the invention including well volumes as low as 0.01 nl.

The use of capillary action of micro-capillary wells open at both ends to draw solution in and to fill the wells, allows the solution to be partitioned into smaller sub-reactions. Further, as the GCA is made of a glass substrate, each micro-capillary well may be made very narrow. The glass substrate may also allow the micro-capillary wells to be packed very closely together to form a high-density array of thousands of wells per square cm. This is because the wall between the wells can be very thin.

Each micro-capillary well may have a high aspect ratio, with capillaries having diameters of 100 urn or less, and depths of 1 mm or more. It will be appreciated that the depths may also be less than 1 mm. This creates very strong capillary action.

Further, the glass substrate is hydrophilic, allowing the solution to be easily drawn into, and holding within, each micro-capillary well through capillary action.

The closely packed micro-capillaries allow a large number of sub-reactions to be created with minimal sample loss in between the wells. This may allow all of the solution to be partitioned, which may allow 100% of the sample to be analyzed in dPCR.

Wells can have high aspect ratio, which gives strong capillary action, and also increases the amount of signal that can be detected after the dPCR (this is because the sensor detects signal along the depth of each well).

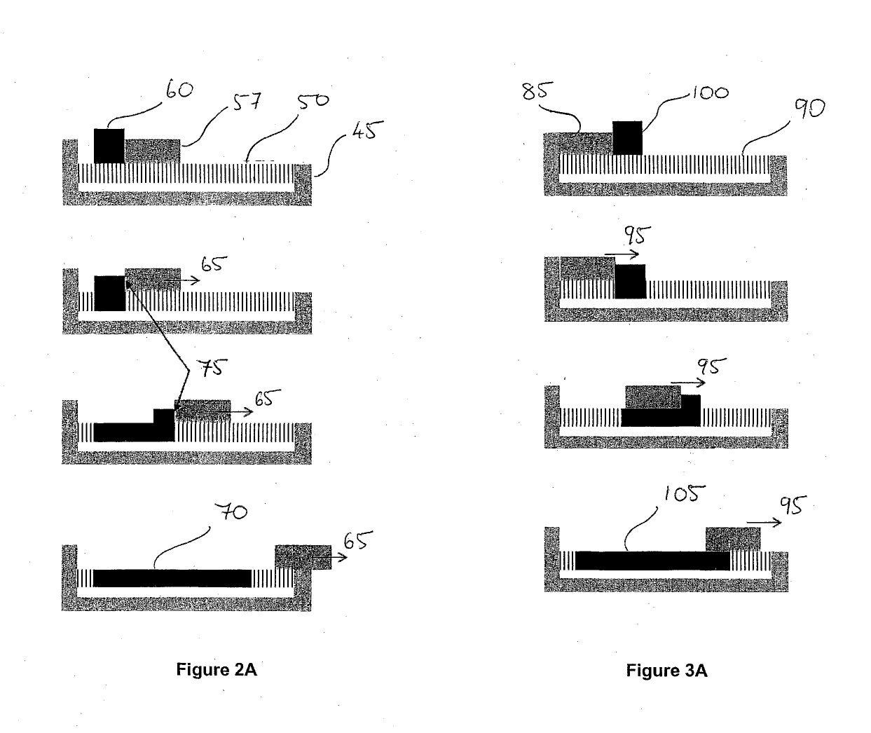

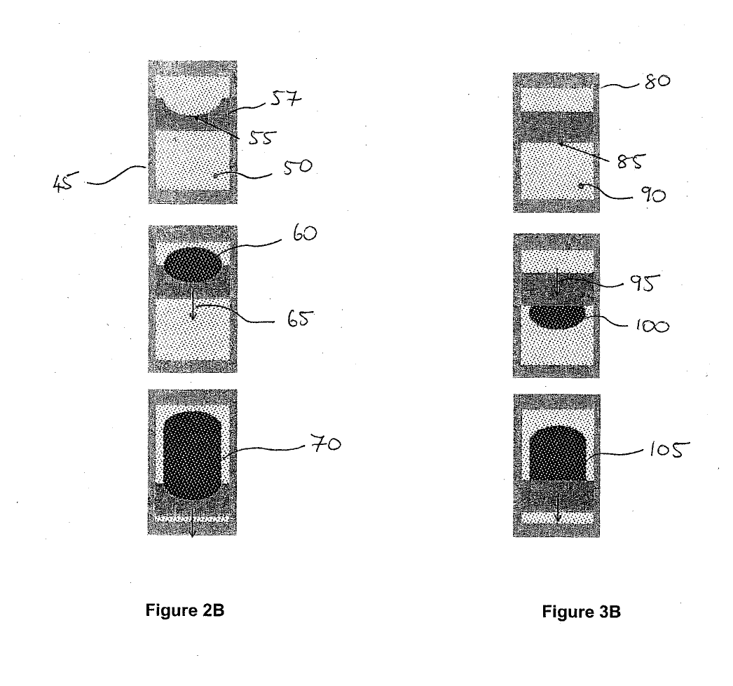

FIGS. 2 and 3 show alternate embodiments directed to facilitating the “draw down” action of the wells.

Normally when a sample 60, 100 is added onto the surface of the GCA 50, 90 and after the wells beneath get filled up, there will still be excess sample. To overcome that, one way is to increase capacity of the well by increasing its depth. However, increasing the volume of each sub-reaction is not desirable for dPCR. Therefore, the sample has to be spread over the GCA, so that all of it gets partitioned into the sub-reactions, without any excess volume. This also allows us to increase the volume of the sample to be added, increasing the number of sub-reactions, which in turn enhances the sensitivity and accuracy of dPCR. Another key aspect of our invention pertains to the method for spreading the sample over the surface of the GCA. Here, we describe the “slider approach”

In this approach, a slider is positioned on top of the GCA. The slider 57, 85 can be positioned such that it is resting on the GCA 50, 90 or there can be a gap between the slider and GCA the sample is added onto a designated area on the GCA. In the embodiment shown in FIGS. 2A and 2B, when the sample is added, it comes into contact with one end of the slider 57. As the slider 57 moves 65 along the GCA 50, it “pulls” the sample along due to the surface tension 75 between the slider and the sample. The idea is to spread 70 the sample along the surface of the GCA, so that the micro-capillary wells get filled up due to capillary action. This is a fast and simple way to fill the GCA, without creating any dead volume. Furthermore, the sample can be dragged along rather fast since the GCA wells fills up rapidly.

The edge of the slider that comes into contact with the sample can be straight or curved 55, or have any other design. In one preferred embodiment, the edge is concaved 55 to increase the contact surface, and hence the surface tension, between the slider and sample. This results in a better “pulling” force as compared to say, a straight edge.

An alternate embodiment is shown in FIGS. 3A and 3B, whereby instead of “pulling” the sample, the slider 85 can also “push” 95 the sample to spread 105 the sample 100 across the GCA 90. In the former, the slider 57 moves over empty wells, whereas in the latter the slider 85 moves over filled wells.

The use of a physical apparatus (i.e. slider) to move the sample over one surface of the GCA, so that it comes into contact with one open end of the micro-capillary wells facilitates more efficient filling of the micro-capillary wells with the sample.

The slider may be made of a hydrophobic material (e.g. PUMA, polycarbonate, or polypropylene) to increase the effectiveness of this method.

In one embodiment, the slider may rest directly on the GCA, without any gap in between. However, there may also be a gap between the slider and GCA.

The edge of the slider that comes into contact with the sample may be curved to increase the pulling force. Through an increase in surface area, consequently increase the attraction force due to surface tension. However, the edge may also be straight for ease of manufacture or another shape for interaction with multiple samples, potential mixing of samples or other such aspect.

Slider should also be of sufficient thickness and width, depending on the volume of sample. Further, the slider may be designed specifically for pushing, pulling or optimized for both subject to technician preference.

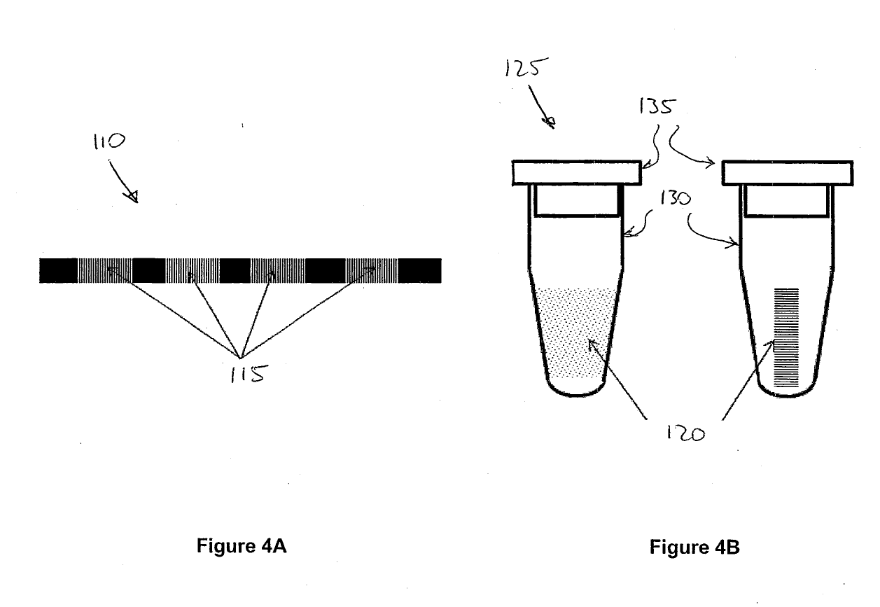

As shown in FIG. 4A, the GCA approach may be configured as a flat chip format 110. Here, multiple GCAs 115 may be integrated onto the chip. Each GCA will have a slider (not shown) positioned over it. This allows the sample to be spread for each GCA. The sliders can either be moved together or individually.

As shown in FIG. 4B, the GCA approach can also be configured as a tube format 125. Here, the GCA 120 is fitted into the interior of a tube 130. The exterior of the tube has the same dimensions of a typical PCR tube, thereby allowing it fit into the heat block of a conventional thermal cycler. The GCA 120 also has a slider (not shown) positioned over it. After the sample has been added, the slider can be moved to spread the sample to fill the GCA, which remains fixed. The tube can be closed by a cap 135. Importantly, the action of closing the cap can provide the same action that causes the slider to spread the sample. Alternatively, the slider may be moved by another action, such as applying an external force to the slide in order to initiate movement. Preferably, the GCA is positioned at or close to the bottom of the tube. This is because most thermal cyclers heat only the bottom part of the PCR tube.

An alternative to the slider approach in spreading the sample over the GCA is to perform centrifugation. The sample must be added to the top of the GCA. Upon centrifugation, the sample is pushed down along the GCA, filling it in the process. This provides a way to move the sample over the GCA without the use of a physical apparatus such as the slider.

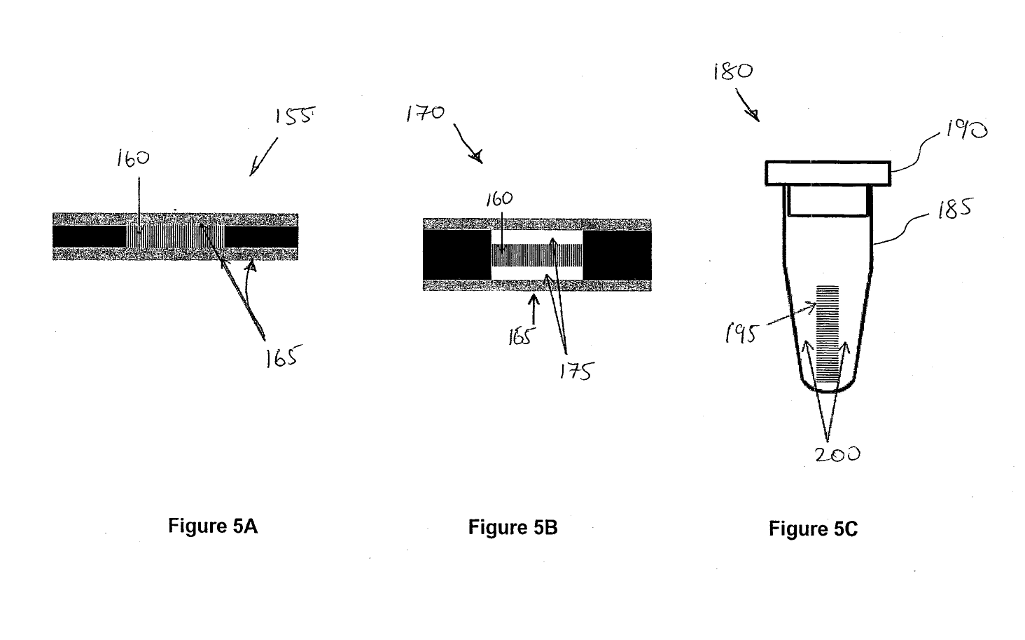

Reference is made to FIGS. 5A to 5C. After the solution has been partitioned into the GCA, PCR is carried out next. During PCR, the heating causes an increase in pressure within the GCA. Since each micro-capillary well is very narrow and the volume within is very small, there is a strong tendency for the solution to be pushed out of the wells. This is undesirable. The solution must be contained within the GCA. To achieve that, one method is to overlay the filled GCA with a mineral oil layer.

FIGS. 5A to 5C demonstrate two alternative methods for holding the sample (or sealing) the solution within the GCA. These form an important aspect of this invention.

In the first approach, as shown in FIG. 5A, the sealing 155 of the sample is achieved by sandwiching the GCA 160 between two thin layers of silicone 165. The two silicone layers 165 are physically pressing onto the GCA 160 to prevent the sample from leaving the wells. The layers can be mechanically pressed onto the GCA 160 by an external force, or it can be due to the adhesive force of the layers itself. In this embodiment, the layers are made of silicone, optically transparent at one or both of the layers. One example is polydimethylsiloxane (PDMS), which can be prepared from a two-part elastomer kit such as Sylgard 184.

It will be appreciated, however, that the layers may be made of other materials also, including glass wafers, polycarbonate, acrylic or other such material suitable for the purpose including mineral oil for at least one of said layers.

In the second approach, as shown in FIG. 5B, we describe the use of “air blankets 175” to seal the sample in the GCA 160. The top and bottom surface of the GCA are sealed by air blankets 175, which are essentially air gaps that become slightly pressurized. The pressurization is formed by keeping the air within a sealed chamber 170.

In the tube format 180, as shown in FIG. 5C, this is achieved by closing the tube with the cap 190—an action that causes slight pressurization 200 of the air within, which helps to sealing the solution in the GCA 195 and prevent it from escaping when heated. Importantly, when the tube gets heated the air within the sealed chamber 185 also gets heated and increases in pressure 200. This further helps to keep the sample in the GCA 195. The advantage of this method is that the sample does not come into contact with any other material, other than the air.

1. A device comprising:
a capillary array of bundled micro-capillary wells;
said bundle arranged into a close packed arrangement;
a first end of said array forming a sample receiving surface;
wherein said device is arranged to draw said sample into said capillary array through capillary action, so as to divide the sample into a plurality of sub-reactions in said wells.

2. The device according to claim 1, further including a distribution tool arranged to slide across said surface to distribute said sample across the surface so as to fill a greater proportion of wells.

3. The device according to claim 2, wherein said distribution tool is arranged to engage the sample through surface tension, pull such that on sliding the tool across the surface said sample is pulled across the surface.

4. The device according to claim 2, wherein said distribution tool is arranged to push the sample across the surface.

5. The device according to claim 2, wherein said distribution tool is in sliding, contact with said surface.

6. The device according to claim 2, wherein said distribution tool is arranged to slide across said surface whilst spaced by a pre-defined gap.

7. The device according to claim 3, wherein said distribution tool includes a sample engagement edge arranged to engage the sample through surface tension, said edge arcuate in shape.

8. The device according to claim 1, wherein said device includes a substrate comprising a plurality of said capillary arrays.

9. The device according to claim 1, further including includes a tube into which the capillary array is placed, said tube selectively sealable with a cap.

10. The device according to claim 1, wherein said glass capillary array is embedded within a chip, said chip sealed with layers proximate to opposed ends of said capillary array.

11. The device according to claim 10, wherein said layers are spaced from the capillary array so as to form an air blanket at said opposed ends

12. The device according to claim 10, wherein said layers are in contact with said opposed ends.

13. The device according to claim 1, wherein the capillary array includes at least 200 wells.

14. The device according to claim 1, wherein the volume of each well within the capillary array is less than or equal to 50 nl.

15. A method of placing a sample within a capillary array, said method comprising the steps of:
placing the sample on a sample receiving surface of said capillary array;
sliding a distribution tool across the sample receiving surface, and so;
distributing the sample across the surface, and consequently;
drawing said sample into wells of said a micro-capillary array through capillary action.

16. The method according to claim 15, wherein the drawing step includes dividing the sample into a plurality of sun-reactions in each well.

17. The method according to claim 15, further including the step, after the drawing step, of placing the capillary array into a chip.

18. The method according to any one of claim 15, further including, the step, after the drawing step, of placing layers over each end of said capillary array so as to seal the wells.

19. The method according to any one of claim 15, further including the steps of placing the capillary array in a tube and sealing said tube.

20-22. (canceled)
